(19) United States
(12) Patent Application Publication
(10) Pub. No.: US 2011/0054279 A1
(43) Pub. Date: Mar. 3, 2011

(54) DIAGNOSIS OF PERIODIC BREATHING

(75) Inventors: Daniel Reisfeld, Tel-Aviv (IL); Yariv Avraham Amos, Hadera (IL); Deganit Barak-Shinar, RaAnana (IL)

(73) Assignee: Widemed Ltd., Herzlia (IL)

(21) Appl. No.: 12/934,672

(22) PCT Filed: Mar. 25, 2009
(86) PCT No.: PCT/IL09/00337

§ 371 Date: Nov. 15, 2010

Related U.S. Application Data
(60) Provisional application No. 61/072,327, filed on Mar. 27, 2008.

(51) Int. Cl. A61B 5/08; A61B 5/145; A61B 5/1455; A61B 5/0205

(52) U.S. Cl. 600/323; 600/529; 600/309; 600/484

(57) ABSTRACT

A method for diagnosis comprises receiving a signal associated with respiration of a patient. The signal is processed so as to identify features that are indicative of beginning and end times of apnea episodes. Time shifts between recurrences of the features are computed and processed so as to generate an output comprising a plurality of bands corresponding to different multiples of a cycle length of the apnea episodes.

### RELATED APPLICATION/S

This application claims the benefit of U.S. Patent Application No. 61/072,327, filed Mar. 27, 2008, the contents of which are hereby incorporated by reference as if fully set forth herein.

### FIELD AND BACKGROUND OF THE INVENTION

The present invention, in some embodiments thereof, relates to physiological monitoring and, more particularly, but not exclusively, to the diagnosis of periodic breathing.

Sleep apneas commonly occur in conjunction with a variety of cardiorespiratory disorders. The relationship between sleep apnea and heart failure, for example, is surveyed by Bradley et al. in two articles entitled “Sleep Apnea and Heart Failure,” including “Part I: Obstructive Sleep Apnea,” Circulation 107, pages 1671-1678 (2003), and “Part II: Central Sleep Apnea,” Circulation 107, pages 1822-1826 (2003), which are incorporated herein by reference. The authors define “apnea” as a cessation of airflow for more than 10 sec. This term is distinguished from “hypopnea,” which is a reduction in but not complete cessation of airflow to less than 50% of normal, usually in association with a reduction in oxyhemoglobin saturation (commonly referred to as “desaturation”).

Sleep apneas and hypopneas are generally believed to fall into two categories: obstructive, due to collapse of the pharynx; and central, due to withdrawal of central respiratory drive to the muscles of respiration. Central sleep apnea (CSA) is commonly associated with Cheyne-Stokes respiration, which is a form of periodic breathing in which central apneas and hypopneas alternate with periods of hyperventilation, with a waxing-waning pattern of tidal volume. CSA is believed to arise as the result of heart failure, though obstructive sleep apnea (OSA) may also occur in heart failure patients.

Both OSA and CSA increase the strain on the cardiovascular system and thus worsen the prognosis of the heart failure patient. In some cases, both types of apneas may occur in the same patient, even at the same time (superposition). Classifying respiratory events as central or obstructive is considered to be a critical point, since treatment may differ according to the type of events, as pointed out by Pepin et al. in “Cheyne-Stokes Respiration with Central Sleep Apnea in Chronic Heart Failure: Proposals for a Diagnostic and Therapeutic Strategy,” Sleep Medicine Reviews 10, pages 33-47 (2006), which is incorporated herein by reference. Both CSA and OSA can be manifested in periodic breathing patterns.

Various methods have been proposed in the patent literature for automated apnea detection and diagnosis based on patient monitoring during sleep. For example, U.S. Patent Application Publication US 2004/0230105 A1 describes a method for analyzing respiratory signals using a Fuzzy Logic Decision Algorithm (FLDA). The method may be used to associate respiratory disorders with obstructive apnea, hypopnea, central apnea, or other conditions. As another example, U.S. Patent Application Publication US 2002/0002327 A1 and U.S. Pat. No. 6,839,581 describe methods for detecting Cheyne-Stokes respiration, which may be used on patients with heart failure. The methods involve performing spectral analysis of overnight oximetry recordings, from which a classification tree is generated. Another method, based on monitoring oxygen saturation and calculating the slope of desaturation events, is described in U.S. Pat. No. 6,760,608. Yet another method for classifying sleep apneas is described in U.S. Pat. No. 6,856,829. In this case, pulse waves from the body of a patient are detected, and the envelope of the pulse waves is created by connecting every peak of the pulse waves. The normalized amplitude and period of the envelope are used in determining whether the patient has OSA, CSA, or mixed sleep apnea syndrome. The disclosures of the patents and patent applications cited above are incorporated herein by reference.

It has been suggested that sleep monitoring can be used for assessing cardiorespiratory risk. For example, U.S. Pat. No. 5,902,250, whose disclosure is incorporated herein by reference, describes a home-based, wearable, self-contained system that determines sleep-state and respiratory pattern, and assesses cardiorespiratory risk. Cardiac disorders (such as cardiac arrhythmia or myocardial ischemia) that are known to be linked to certain respiratory disorders also may be inferred upon detection of such respiratory disorders.

Photoplethysmograph devices, known commonly as pulse oximeters, provide instantaneous in vivo measurement of arterial oxygenation by determining the color of blood between a light source and a photodetector. To determine the blood oxygen saturation, light absorption measurement is carried out at two wavelengths in the red and infrared ranges. The difference between background absorption during diastole and peak absorption during systole at both wavelengths is used to compute the blood oxygen saturation.

Photoplethysmograph signals provide information not only on blood oxygenation, but also on other physiological signs. For example, U.S. Pat. No. 5,588,425 describes the use of a pulse oximeter in validating the heart rate and/or R-R intervals of an ECG, and for discriminating between sleep and wakefulness in a monitored subject. It also describes a method for distinguishing between valid pulse waveforms in the oximeter signal. U.S. Pat. No. 7,001,337 describes a method for obtaining physiological parameter information related to respiration rate, heart rate, heart rate variability, blood volume variability and/or the autonomic nervous system using photoplethysmography. US Patent Application Publication 2007/0213622 describes a method for diagnosis that includes receiving a signal, such as a photoplethysmograph signal, that is associated with blood oxygen saturation of a patient during sleep. The signal is filtered and processed to detect a pattern corresponding to multiple cycles of periodic breathing. The disclosures of the above-mentioned patents and patent applications are incorporated herein by reference.

### SUMMARY OF THE INVENTION

Embodiments of the present invention that are described hereinbelow provide improved methods, systems and software for detection and analysis of periodic breathing patterns. The disclosed techniques permit such patterns to be visualized for rapid diagnosis and generate a prognostic indicator of heart disease with high sensitivity and specificity.

There is therefore provided, in accordance with an embodiment of the present invention, a method for diagnosis, including receiving a signal associated with respiration of a patient. The signal is processed so as to identify features that are indicative of beginning and end times of apnea episodes. Time shifts between recurrences of the features are computed and processed so as to generate an output including a plurality of bands corresponding to different multiples of a cycle length of the apnea episodes.

In some embodiments, the signal is indicative of variations in a blood oxygen saturation in a body of the patient due to the apnea episodes. In one embodiment, the signal is received from a photoplethysmographic sensor coupled to the body of the patient. Additionally or alternatively, processing the signal includes defining a threshold level, and identifying the features as points at which the blood oxygen saturation crosses the threshold level.

In a disclosed embodiment, processing the time shifts includes presenting a plot of the bands on a display, wherein presenting the plot includes displaying the time shifts as a function of time over a monitoring period. Additionally or alternatively, processing the time shifts includes generating a histogram of the time shifts, wherein processing the time shifts includes fitting a parametric function to the histogram so as to extract the cycle length from the histogram. The parametric function may include a sum of kernel functions.

The method may include including diagnosing a medical condition of the patient responsively to the cycle length, wherein the medical condition includes a heart failure of the patient, and wherein diagnosing the medical condition includes assessing a severity of the heart failure based on the cycle length.

There is also provided, in accordance with an embodiment of the present invention, apparatus for diagnosis, including a sensor, which is configured to be coupled to a body of a patient and to output a signal associated with associated with respiration of the patient. A processor is coupled to process the signal so as to identify features that are indicative of beginning and end times of apnea episodes, to compute time shifts between recurrences of the features, and to process the time shifts so as to generate an output including a plurality of bands corresponding to different multiples of a cycle length of the apnea episodes.

There is additionally provided, in accordance with an embodiment of the present invention, a computer software product, including a computer-readable medium in which program instructions are stored, which instructions, when read by a computer, cause the computer to receive a signal associated with respiration of a patient, to process the signal so as to identify features that are indicative of beginning and end times of apnea episodes, to compute time shifts between recurrences of the features, and to process the time shifts so as to generate an output including a plurality of bands corresponding to different multiples of a cycle length of the apnea episodes.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A and 1B are schematic, pictorial illustrations of a system for sleep monitoring and diagnosis, in accordance with an embodiment of the present invention;

FIG. 2 is a schematic plot of a photoplethysmograph signal, illustrating a method of processing such signals in accordance with an embodiment of the present invention;

FIGS. 3, 5 and 7 are plots that schematically show patterns of recurring features in blood oxygen saturation signals as a function of time shift, in accordance with an embodiment of the present invention;

FIGS. 4, 6 and 8 are plots that schematically show histograms of the patterns of FIGS. 3, 5 and 7, respectively, in accordance with an embodiment of the present invention;

FIG. 9 is a receiver operating characteristic (ROC) plot, which schematically shows the sensitivity and specificity of predicting heart failure prognosis in accordance with an embodiment of the present invention; and

FIG. 10 is a Kaplan-Meier plot that schematically shows prognosis of heart failure patients as a function of the severity of symptoms classified by a method in accordance with an embodiment of the present invention.

FIG. 11A exemplifies a raw saturation signal as a function of the time in minutes, as acquired according to various exemplary embodiments of the present invention.

FIG. 11B shows the signal of FIG. 11A following a noise reduction preprocessing performed according to some embodiments of the present invention.

FIG. 12A exemplifies a derivative signal as extracted from a saturation signal following a noise reduction preprocessing, according to various exemplary embodiments of the present invention.

FIG. 12B exemplifies a saturation signal in which desaturation intervals have been marked based on the derivative signal of FIG. 12A, according to various exemplary embodiments of the present invention.

FIG. 12C shows the same signal as FIG. 12B, following an saturation reduction thresholding procedure in which a 3% saturation reduction threshold is employed, according to some embodiments of the present invention.

FIG. 12D shows the same derivative signal as FIG. 12B, following an saturation reduction thresholding procedure in which a 4% saturation reduction threshold is employed, according to some embodiments of the present invention.

FIG. 13 depicts a high level block diagram of a prototype system, according to some embodiments of the present invention.

FIG. 14 further details a saturation unit of the prototype system of FIG. 13, according to some embodiments of the present invention.

FIG. 15A shows example of saturation during Cheyne Stokes breathing, as acquired according to some embodiments of the present invention.

FIG. 15B illustrates a parametric model fitting of 10 minutes of the Cheyne Stokes of FIG. 15A, using a Gaussian kernel function, according to some embodiments of the present invention.

FIGS. 16A-B present a chaotic effect of obstructive sleep apnea, as acquired, analyzed and presented according to some embodiments of the present invention.

### DESCRIPTION OF SPECIFIC EMBODIMENTS OF THE INVENTION

The present invention, in some embodiments thereof, relates to physiological monitoring and, more particularly, but not exclusively, to the diagnosis of periodic breathing.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### System Overview

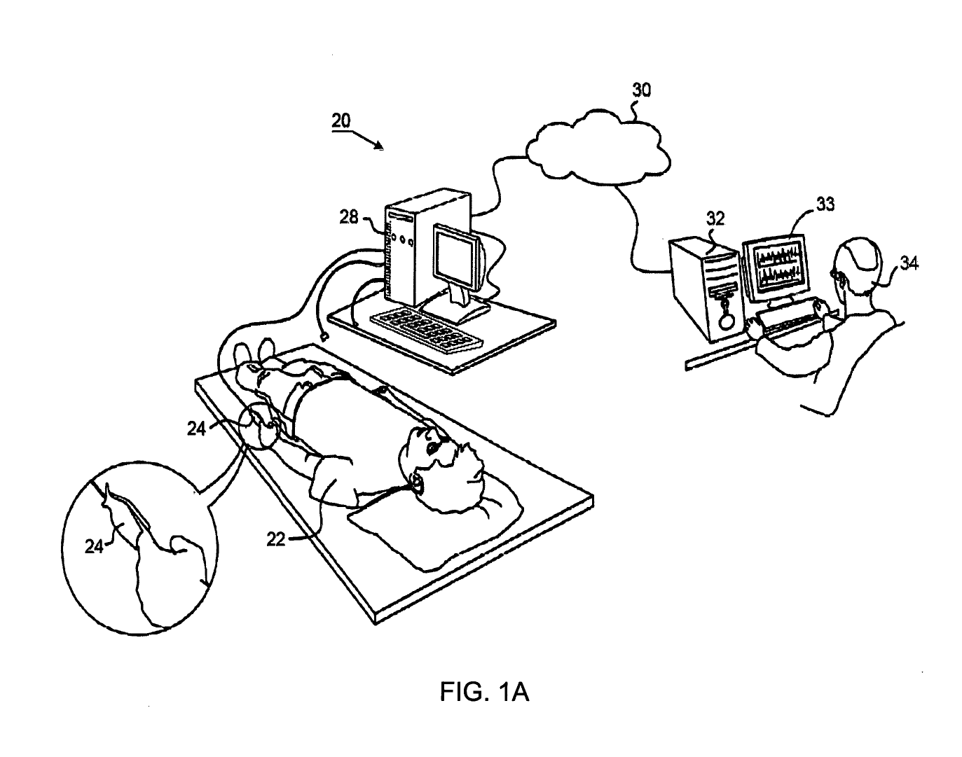

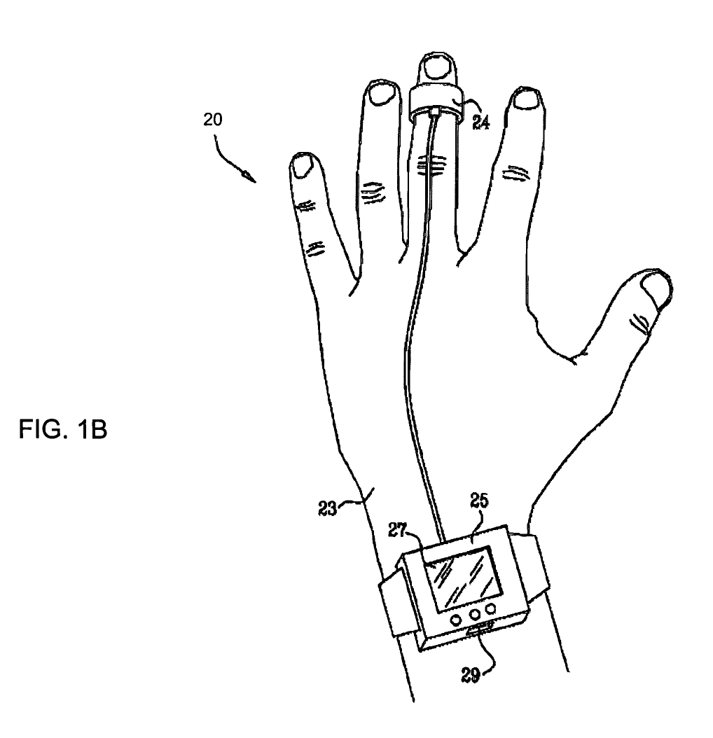

FIGS. 1 and 1B are schematic, pictorial illustrations of a system 20 for sleep monitoring and diagnosis, in accordance with an embodiment of the present invention. In this embodiment, system 20 is used to monitor a patient 22 in a home, clinic or hospital ward environment, although the principles of the present invention may similarly be applied in dedicated sleep laboratories.

System 20 receives and analyzes a photoplethysmograph signal from a suitable sensor, such as a pulse oximetry device 24. Device 24 provides photoplethysmograph signals indicative of blood flow and of the level of oxygen saturation in the patient's blood, which is in turn dependent on respiration. In the context of the present patent application and in the claims, the photoplethysmograph signal is thus considered to be a signal that is associated with blood oxygen saturation and with respiration. Since the photoplethysmograph signal is modulated by both the heart rate and respiratory rate, it may also be used to provide a heart rate and respiratory rate signals. The sensor signals from device 24 are collected, digitized and processed by a console 28.

Pulse oximetry device 24 may have the form of a ring, which fits comfortably over one of the fingers on the hand of the patient (see FIG. 1B). Other configurations of device 24 are not excluded from the scope of the present invention. Device 24 can be connected by a wire to a control unit 25, which may conveniently be fastened around the patient's wrist. Alternatively, the control unit may be fastened elsewhere on the patient's forearm, at any suitable location between the hand and the elbow, or elsewhere on the patient's body. The control unit may include a display, to present status information and/or readings of monitored parameters, such as heart rate, blood oxygen saturation and heart failure status. A connector 29 on the control unit is configured to connect to a console or docking station. In the illustrated embodiment, connector 29 comprises a receptacle for a cable with a standard plug, such as a USB cable. Alternatively, the connector may mate directly with a matching connector on a dedicated docking station.

Additionally or alternatively, system 20 may comprise sensors of other types (not shown), for collecting other physiological signals. For example, the system may receive an ECG signal, measured by skin electrodes, and a respiration signal measured by a respiration sensor. Additionally or alternatively, the techniques of monitoring and analysis that are described herein may be combined with EEG, EOG, leg motion sensors, and other sleep and/or cardiac monitoring modalities that are known in the art. As another example, console 28 may receive signals by telemetry from implantable cardiac devices, such as pacemakers and ICDs.

Console 28 may process and analyze the signals from pulse oximetry device 24 locally, using the methods described hereinbelow. In the present embodiment, however, console 28 is coupled to communicate over a network 30, such as a telephone network or the Internet, with a diagnostic processor 32. This configuration permits sleep studies to be performed simultaneously in multiple different locations. Processor 32 typically comprises a general-purpose computer processor (which may be embedded in a bedside or remote monitor) with suitable software for carrying out the functions described herein. This software may be downloaded to processor 32 in electronic form, or it may alternatively be provided on tangible media, such as optical, magnetic or non-volatile electronic memory. Processor 32 analyzes the signals conveyed by console 28 in order to analyze the physiological parameters and extract prognostic information regarding patient 22. The processor outputs the results of the analysis to an operator 34, such as a physician, via an output device such as a display 33. Alternatively, processor 32 can comprise a special computer processor configured for carrying out the functions described herein. For example, processor 32 can be a special computer processor which comprises special firmware embodying computer instructions for carrying out the functions described herein.

While the embodiments herein are described with a particular emphasis to methods and apparatus for monitoring and diagnosis during sleep, it is to be understood that more detailed reference to diagnosis during sleep is not to be interpreted as limiting the scope of the invention in any way. The principles of the present invention may also be applied, mutatis mutandis, to patients who are awake. For example, these methods and apparatus may be used in monitoring patients who are reclining or otherwise at rest, even if they are not asleep.

The configuration of system 20 that is shown in FIG. 1 is described here by way of example, and the principles of the present invention may likewise be applied in other patient monitoring configurations. Some applicable configurations are shown, for example, in the above-mentioned US 2007/0213622. Furthermore, although the inventors have found it convenient to collect and process photoplethysmograph signals as the basis for diagnosis of periodic breathing patterns, the principles of the present invention may similarly be applied to other types of physiological signals that are indicative of patient respiration.

For example, system 20 and processor 32 may be configured to collect and process electrical measurements of thoracic and abdominal movement, using skin electrodes to make a plethysmographic measurement of the patient's respiratory effort or a belt to sense changes in the body perimeter. Additionally or alternatively, air flow measurement, based on a pressure cannula, thermistor, or CO₂ sensor, may be used for respiration sensing. In other embodiments of the present invention, other sensors that are known in the art of polysomnography, such as a capnograph, may be used in detecting sleep apneas, either in conjunction with or separately from the pulse oximeter used in the techniques described above. In yet another embodiment, an electrocardiogram (ECG) signal may be used in detecting apneas, since the ECG signal also exhibits changes that are associated with respiration.

### Signal Processing and Display

In the description that follows, techniques for processing respiration-related signals and displaying processing results are described, for convenience and clarity of explanation, with reference to system 20, as shown in FIGS. 1A and 1B. These techniques, however, are in no way limited to this particular system, and they may likewise be applied in substantially any suitable system configuration for collection and processing of respiration-related signals.

In various exemplary embodiments of the invention pulse oximetry device 24 generate signals that are indicative of absorption and/or reflectance of light at two different wavelengths, typically one red and one infrared, as is known in the art. Each signal includes an AC component, which corresponds to the pulsatile change in the signal at the patient's heart rate, and a slow-changing DC component. Comparison of the AC components of the two signals gives a blood oxygen saturation signal. Alternatively, at least some of the methods described below can use the signals from only a single source/detector subassembly, or signals provided by other types of sensors, as further detailed hereinabove.

In some embodiments of the present invention the saturation signal is preprocessed. In some embodiments, the preprocessing includes low-pass filtration so as to provide a very-low-frequency (VLF) saturation signal. This filtering removes signal components at frequencies that are greater than or equal to the patient's respiratory frequency, such that the remaining signal reflects trends over multiple respiratory cycles. In some embodiments, the filtering is even more pronounced, and eliminates frequency components outside the Cheyne-Stokes cycle frequency, for example, components below 1/180 Hz or above 1/40 Hz. In some embodiments of the present invention the preprocessing includes identification and removal of noisy events and post-removal signal reconstruction, as further exemplified in the Examples section that follows.

Processor 32 analyzes shape characteristics of the optionally filtered saturation signal in order to detect episodes of Cheyne-Stokes breathing (CSB). This condition is characterized by a regular waxing and waning breathing pattern and occurs particularly among patients with heart failure and in patients who have experienced a stroke. CSB is present during sleep, and in more severe cases may also be observed during wakefulness. According to the American Academy of Sleep Medicine, Cheyne-Strokes breathing syndrome (CSBS) is characterized by the following criteria: (1) presence of congestive heart failure or cerebral neurological disease; and (2) respiratory monitoring demonstrates: (a) at least three consecutive cycles of a cyclical crescendo and decrescendo change in breathing amplitude, where cycle length is most commonly in the range of 60 seconds, although the length may vary, and (b) one or both of the following: (i) five or more central sleep apneas or hypopneas per hour of sleep, and (ii) the cyclic crescendo and decrescendo change in breathing amplitude has duration of at least 10 consecutive minutes.

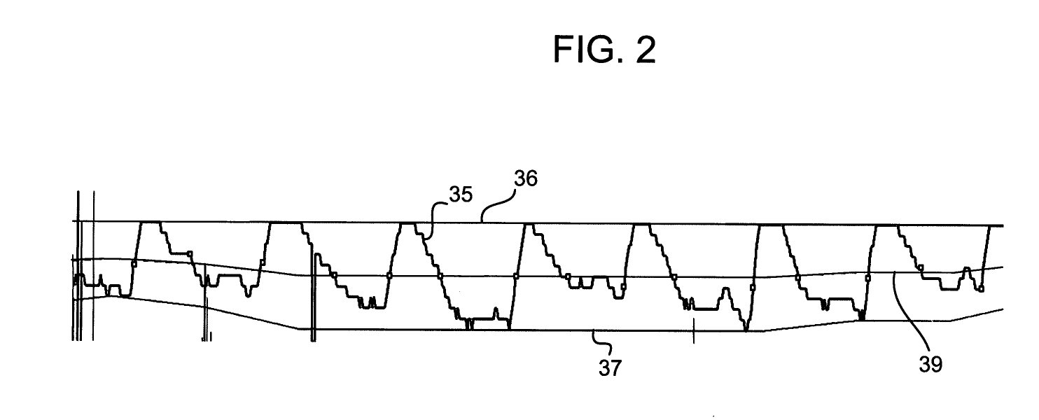

FIG. 2 is a schematic plot of a raw saturation signal 35, illustrating a method of processing such signals in accordance with an embodiment of the present invention. The signal shown in the figure gives a measure of blood oxygen saturation over time during sleep of a patient suffering from recurrent apneas. In some embodiments of the present invention processor 32 analyzes signal 35 to find and mark the times of occurrence of signal features that are indicative of the beginning and end of apnea episodes. For this purpose, a processor identifies local maxima and minima in signal 35. In some embodiments, processor identifies the highest local maximum and lowest local minimum within a given sliding window (for example, once every three minutes). Thus, upper and lower lines 36 and 37 represent the positions of the highest local maximum and lowest local minimum of signal 35.

In some embodiments of the present invention processor 32 takes the average of the upper and lower lines as a threshold level 39. In these embodiments, the processor identifies each intersection of level 39 by the saturation curve of signal 35 as a feature time, and records the time as either a descending (beginning of apnea) or ascending (end of apnea) feature type. To ensure that the features represent actual apneas, the processor records only features that represent at least a certain minimal change in the saturation level (for example, at least 4% desaturation). The record of feature occurrences as a function of time thus has the form of a binary signal, with transitions between the signal levels corresponding to the zero-crossing times.

In some embodiments of the present invention mathematical derivatives are employed for identifying features indicative of apnea episodes. This can be done, for example, by extracting the cumulative sum of the first derivative of the signal (e.g., saturation signal), so as to provide a derivative signal. In embodiments in which preprocessing is employed, the cumulative sum of the first derivative is preferably extracted after the preprocessing. The cumulative sum is preferably calculated by summing the values of the derivatives at sequential sampling time points. A change of sign of the cumulative sum indicates the end of an episode.

Once the derivative signal is provided, a “feature space” is spanned by two or more properties of the derivative signal and optionally the signal itself (preferably after preprocessing). The feature space is the working space for the purpose of classifying the features and identifying features indicative of apnea episodes, as further detailed hereinunder. Representative examples of properties suitable for spanning the feature space include, without limitation, slope, duration and desaturation level.

In some embodiments of the present invention, the spanning of feature space is preceded by a procedure in which two or more intervals over the derivative signal are unified (i.e., considered as one interval although being spaced apart) according to a predetermined criterion or set of criteria. Preferably, the unification procedure is executed for unifying intervals characterized by non-positive changes of the derivative signal. In some embodiments of the present invention, the criteria for unifying two such intervals are; (i) the distance between two events is less than a predetermined duration (say, 20 seconds), and (ii) the overall saturation reduction of each of the two intervals is sufficiently low compared to adjacent desaturation episodes (e.g., less than twice the saturation reduction thereof).

Once the feature space is spanned, features indicative of apnea episodes are preferably identified by applying a saturation reduction thresholding procedure. In various exemplary embodiments of the invention a feature is identified as indicative of an apnea episode if the overall saturation reduction over the time intervals associated with the feature is at least a predetermined saturation reduction threshold (for example, at least 3% or at least 4% desaturation).

The identified features are further analyzed according to distances between starts and end of events so as to produce periodic respiratory breathing event, as further detailed hereinunder.

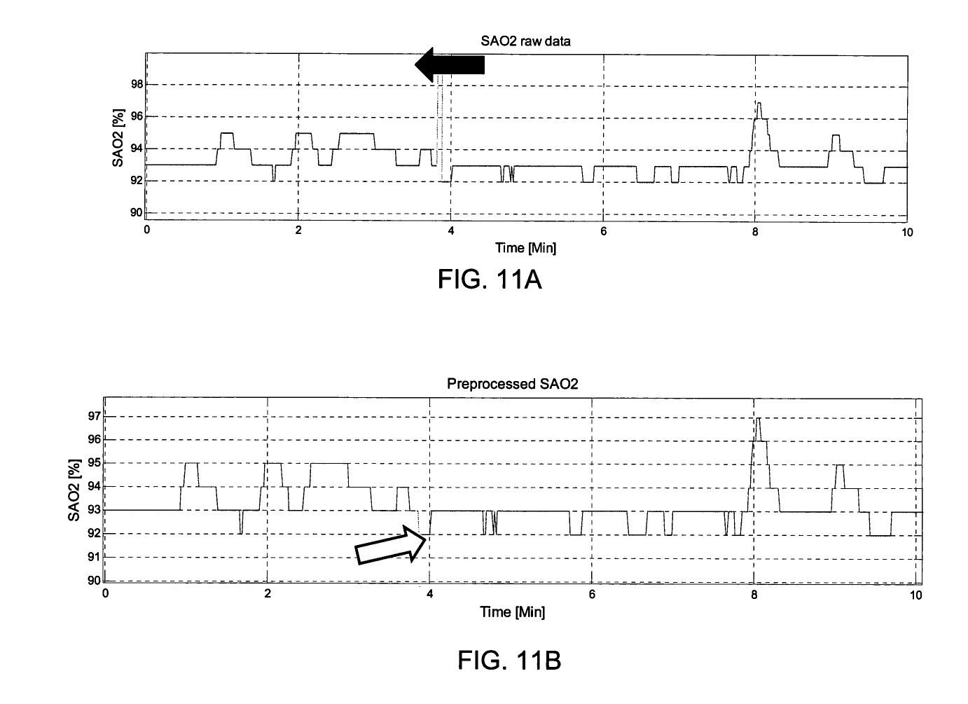

FIGS. 11A-B and 12A-D exemplify selected operations of the above procedure.

FIG. 11A exemplifies a raw saturation signal (as a function of the time in minutes), and FIG. 11B shows the same signal following a noise reduction preprocessing. In the exemplifies signal, there is one noisy peak (shown by a black block arrow in FIG. 11A) which has been removed. Once the noisy peak is removed, the signal is reconstructed by adding a synthetic insert so to provide a substantially smooth signal. The synthetic insert is shown by white block arrow in FIG. 11B.

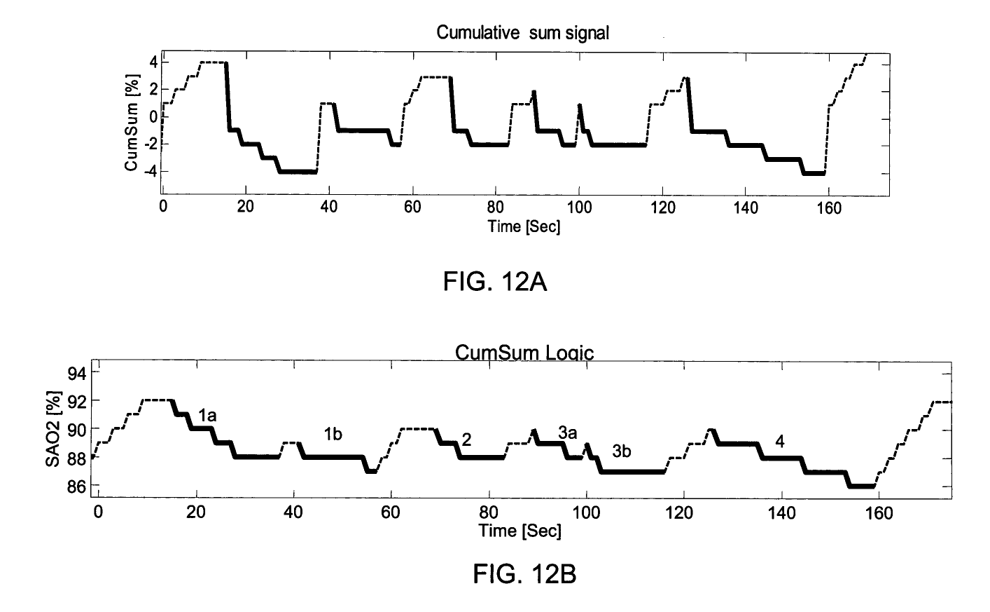

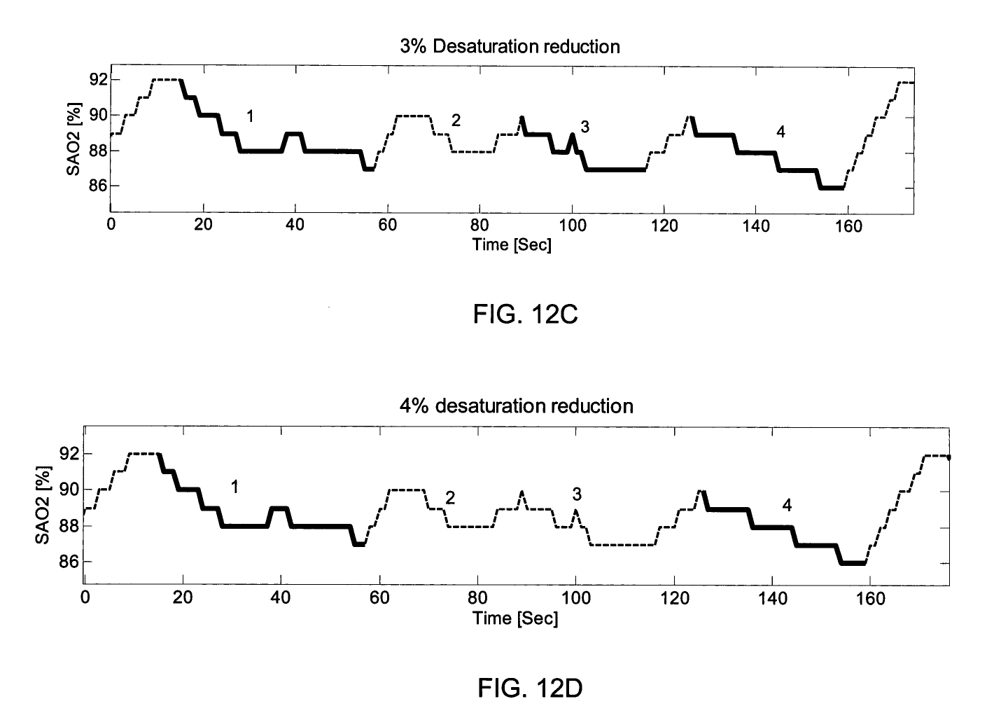

FIG. 12A exemplifies a derivative signal as extracted from a saturation signal following a noise reduction preprocessing. Intervals of positive derivative are shown as dash lines and intervals of non-positive derivative are shown as solid lines.

FIG. 12B exemplifies a saturation signal in which desaturation intervals have been marked based on the derivative signal of FIG. 12A. The desaturation intervals are marked as solid lines while other intervals are marked as dash lines. Several representative desaturation intervals are marked on FIG. 12B by numerals 1a, 1b, 2, 3a, 3b and 4. Intervals 1a and 1b satisfy the unification criteria and are therefore treated as a single interval, referred to hereinunder and in FIGS. 12C-D as interval 1. Likewise, intervals 3a and 3b satisfy the unification criteria and are therefore treated as a single interval, referred to hereinunder and in FIGS. 12C-D as interval 3.

FIG. 12C shows the same derivative signal as FIG. 12B, following an saturation reduction thresholding procedure in which a 3% saturation reduction threshold is employed. Time intervals identified as corresponding to apnea episodes are shown as solid lines. All other intervals are shown as dash lines. As shown, interval 4 as well as the unified intervals 1 and 3 (which are the unification of intervals 1a, 1b and 3a, 3b, respectively) are identified as corresponding to apnea episodes. Interval 2 is excluded since its overall desaturation is less than 3%.

FIG. 12D shows the same derivative signal as FIG. 12B, following an saturation reduction thresholding procedure in which a 4% saturation reduction threshold is employed. Time intervals identified as corresponding to apnea episodes are shown as solid lines. All other intervals are shown as dash lines. As shown, interval 1 and 4 are identified as corresponding to apnea episodes. Intervals 2 and 3 are excluded since their overall desaturation is less than 4%.

Processor 32 computes the time shifts between recurrences of features in this binary signal over time in order to detect periodic patterns, which are indicative of periodic breathing. This recurrence function may be computed quickly and simply using logical and shift operations.

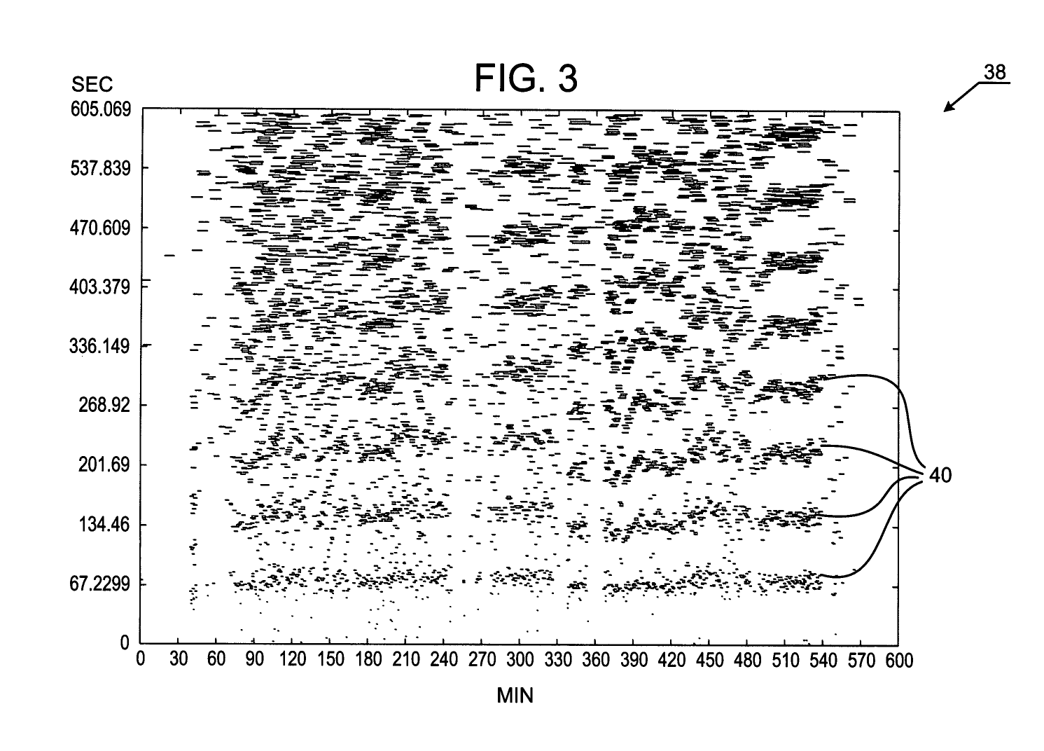

FIG. 3 is a schematic plot 38 of the recurrence function of a saturation signal as a function of time shift, in accordance with an embodiment of the present invention. The patient in this case was independently diagnosed as suffering from Cheyne-Stokes breathing syndrome. The horizontal axis in FIG. 3 corresponds to time (in minutes) from the beginning of a monitoring session, while the vertical axis represents time shift (in seconds). For each feature (zero-crossing) occurring at a given time on the X-axis, processor 32 searches backward in time for previous occurrences of the same type of feature (ascending or descending). Each point marked in plot 38 indicates a recurrence of the feature of time X at a shift of time Y.

Plot 38 shows a clear pattern of bands 40, meaning that recurrences of features in the saturation signal occurred with a consistent, periodic cycle length (in this case, approximately 67 sec). The inventors have found this band structure to be characteristic of Cheyne-Stokes breathing, with each band corresponding to a different multiple of the cycle length. This sort of plot may be presented by system 20 on display 33 and enables operator 34 to visually diagnose the condition of patient 22. The period and strength of the bands appearing on the display give a good indication of the severity of the patient's condition, which is immediately apparent to the operator.

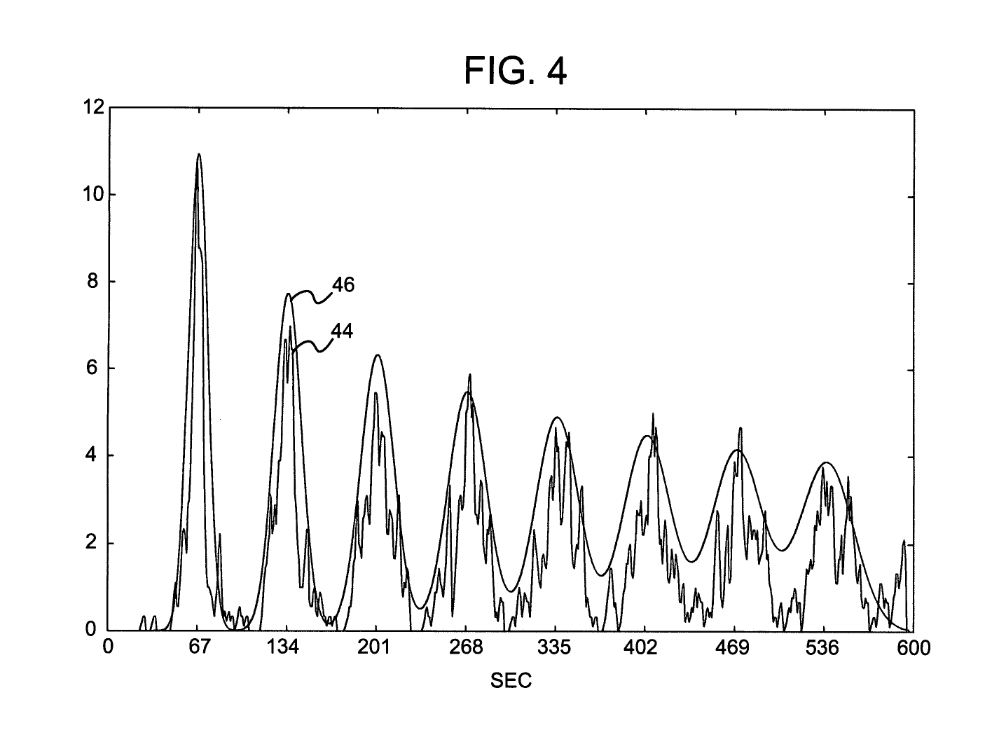

FIG. 4 is a plot that schematically shows a histogram 44 of the recurrence function of FIG. 3, in accordance with an embodiment of the present invention. The histogram compiles the number of recurrence points in the recording of FIG. 3 as a function of the time shift, and can be seen to comprise multiple bands corresponding to multiples of the basic cycle length. The histogram may likewise be presented on display 33 in order to give the operator another diagnostic indicator.

Additionally or alternatively, processor 32 may fit a parametric model 46 to histogram 44 in order to automatically extract the period and amplitude of the histogram. In various exemplary embodiments of the invention histogram 44 is fitted to an analytical function CS(Δ_{f}) representing the probability of finding a pair of features Δ_{f} seconds apart. For example, CS(Δ_{f}) can be calculated using a set of kernel functions K, (i=1, 2, . . . , n). Preferably, CS(Δ_{f}) is calculated a sum, e.g., using the following equation

CS(Δ_{f})=ΣᵢwᵢKᵢ

where wᵢ is the weight of the ith kernel function. The number n of kernel functions in the fit can be predetermined or it can be a free parameter which is selected to optimize the goodness of the fit. In various exemplary embodiments of the invention at least one, more preferably all the kernel functions, is a localized function.

As used herein, “localized function” refers to any function having a local support and which is significantly suppressed far (e.g., at a distance of about 10 widths of the local support) from the local support. Representative examples of localized functions include, without limitation, a Gaussian function, a Lorentzian function, a hyperbolic secant function (also known as sech), a logistic distribution and the like. Additionally, the localized function can be represented as a series or an integral of other functions. For example, the localized function can be a Fourier transform, a wavelet transform and the like.

In embodiments in which the kernel functions are Gaussians, the following expression can be used for the analytical function CS(Δ_{f}):

$${CS}_{λ , σ} ( {Δ}_{t} ) = \frac{a}{\sqrt{2 π} σ} {\sum }_{i = 1}^{n} \frac{1}{\sqrt{i}} {e}^{- \frac{{( {Δ}_{t} - iλ )}^{2}}{2 {iσ}^{2}}} ,$$

where, α is an amplitude factor, λ is the average distance between consecutive apneas; σ is the standard deviation of the distance between consecutive apneas, and the summation is taken over multiples i of the average distance λ. The parameters α, λ, σ and optionally n are preferably selected to optimally fit the histogram of distances between features.

As can be seen in FIG. 4, model 46 in this case is strongly periodic, with a cycle length of about 67 sec, and with a high amplitude and low standard deviation (substantially less than the period of the pattern). These features thus give a rapid quantitative assessment of the patient's respiratory conditions.

In some embodiments of the present invention processor 32 evaluates the slope of the saturation signal (or of the DC component of the pulse oximeter signal) for each desaturation episode. This is particularly useful for ensuring that the sequences of cyclic breathing episodes are indeed associated with the severity of heart failure status. In central apnea, or when the heart failure state is grave, the slope of the exit from the cycle is moderate, e.g., similar to the typical (or specific) entry slope. Therefore, to identify a time sequence of cyclic breathing as Cheyne-Stokes, processor 32 requires that the sequence comprise mainly (typically at least 80%) events of moderate slope. (PCT Patent Application PCT/IL2006/000148, the contents of which are hereby incorporated by reference, defines formal criteria for assessing the symmetry of periodic breathing episodes, which may also be used in the present context for distinguishing Cheyne-Stokes events.). The requirement of moderate slope may also be applied to the median slope value.

The observations with respect to the symmetry of the periodic breathing patterns apply both to the slowly-varying heart rate and saturation signals and to the envelopes of the other, rapidly-varying signals shown by the other traces. The term “envelope” in this context typically means a signal derived from the local minima and/or local maxima of another signal, with or without smoothing (by convolution or resampling, for example). “Envelopes” may also be derived by other mathematical operations known in the art, such as application of Hilbert transforms. The inventors have found that periodic breathing patterns associated with CSA generally tend to be more symmetrical than the patterns associated with OSA, presumably due to the different physiological mechanisms that are involved in the different types of apneas. Therefore, processor 32 may validate the prognostic value of the Cheyne-Stokes marker by considering only events with mild exit slope from desaturation events. The inventors found that computing the slope of the saturation curve by fitting a line (by the least-square method) to the curve over a nine-second epoch, and requiring that the slope of the line be less then 0.7 percent/second is a good implementation of this mild desaturation condition.

The features of the Cheyne-Stokes segments are prognostic of patient outcome in cases of heart failure (and other illnesses). In particular, long wavelength is associated with bad prognosis. Thus, processor 32 typically detects signal components that have a period greater than a minimum period of at least 30 sec. For example, a periodic breathing pattern is classified as a Cheyne-Stokes breathing if the median cycle length is above 55 seconds and the median desaturation value is at least 2%. or at least 3% or at least 4%.

On the other hand, time segments with steep exit saturation slope typically correspond to obstructive apnea/hypopnea events. Other features of obstructive apnea/hypopnea time segments include short wavelength, large vasomotion, and large heart rate modulations. These phenomena are generally associated with good prognosis, since they reflect the patient's ability to manifest enhanced sympathetic activity.

In addition to the saturation measurements and Cheyne-Stokes detection, processor 32 may also process an AC absorption or reflectance signal that is output by device 24 in order to compute a heart rate, as is known in the art. The AC signal may also be analyzed to detect a beat morphology. The processor can identify certain aberrations in this morphology as arrhythmias, such as premature ventricular contractions (PVCs). The processor optionally keeps a record of the occurrences of such arrhythmias, in a manner similar to a Holter monitor, but without requiring the use of ECG leads. The total number of abnormal heart beats and (specifically PVCs) that are accumulated in such a record, particularly during sleep, is indicative of bad prognosis. As the inventors have found that premature beats during sleep have the greatest prognostic value for advanced heart failure patients, the processor may be configured to count the number of premature beats only during sleep or during episodes of Cheyne-Stokes breathing.

The inventors have found typical Cheyne-Stokes cycle lengths to be between 40 and 90 sec. The cycle length, as well as the other parameters, depends on the severity of the patient's heart failure. In particular, the inventors have found that decompensated heart failure patients nearly always present long sequences of periodic Cheyne-Stokes breathing episodes, with a cycle length between 55 and 180 seconds. In general, the longer the cycle length, the more severe is the state of the disease. Therefore, processor 32 may use the time characteristics of the patient's Cheyne-Stokes episodes as a prognostic indicator.

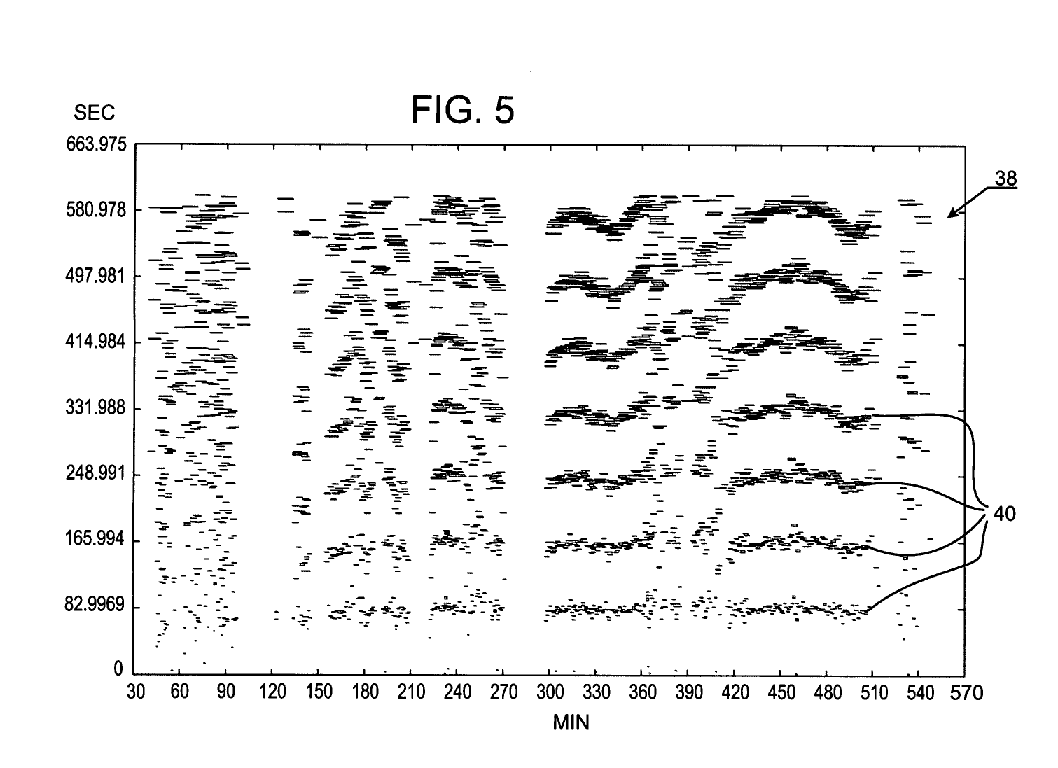

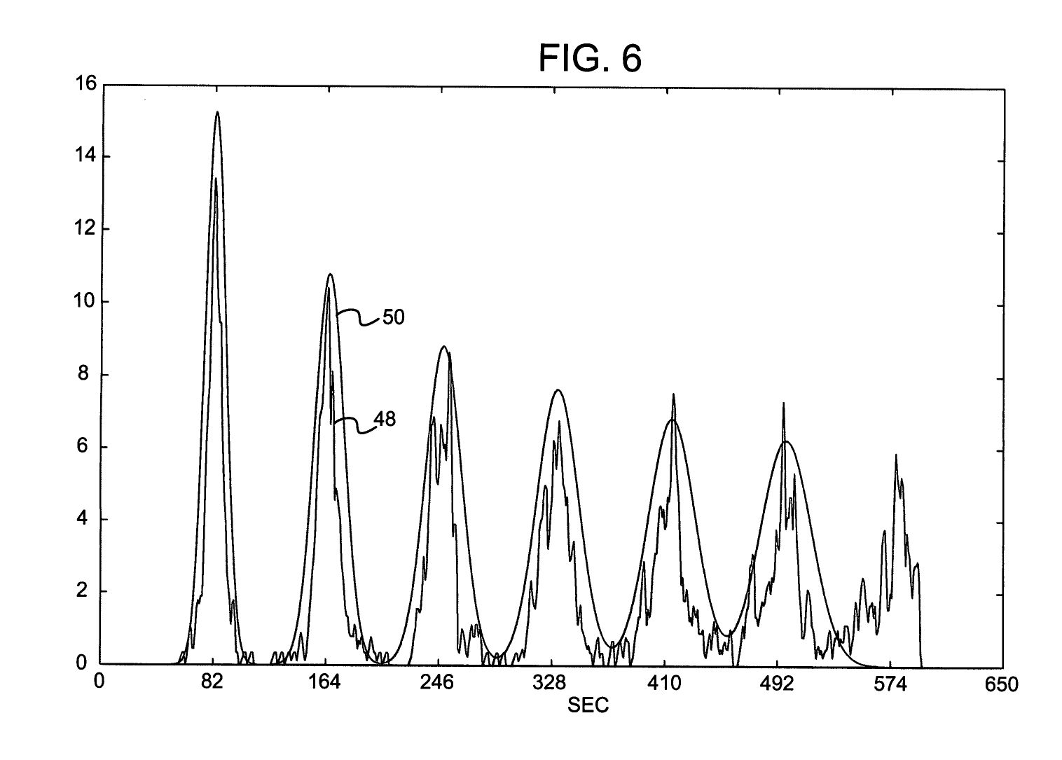

FIGS. 5 and 6 are schematic plots of the recurrence function and histogram, respectively, of a saturation signal in another patient, in accordance with an embodiment of the present invention. The patient in this case suffered from more severe heart failure than the patient of FIGS. 3 and 4, and the Cheyne-Stokes bands 40 in FIG. 5 are therefore sharply marked. The severity of the patient's condition is reflected in a highly-periodic histogram 48 of the recurrence function. A parametric curve 50, fitted to histogram 48 using the equation shown above, indicates that the Cheyne-Stokes cycle length in this case is about 82 sec.

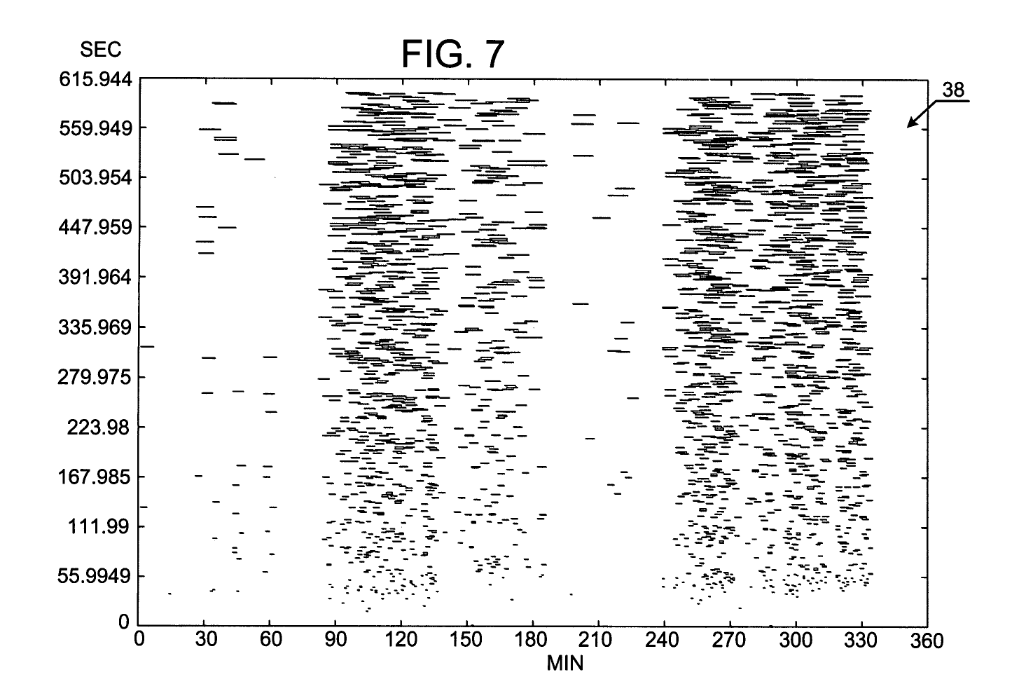

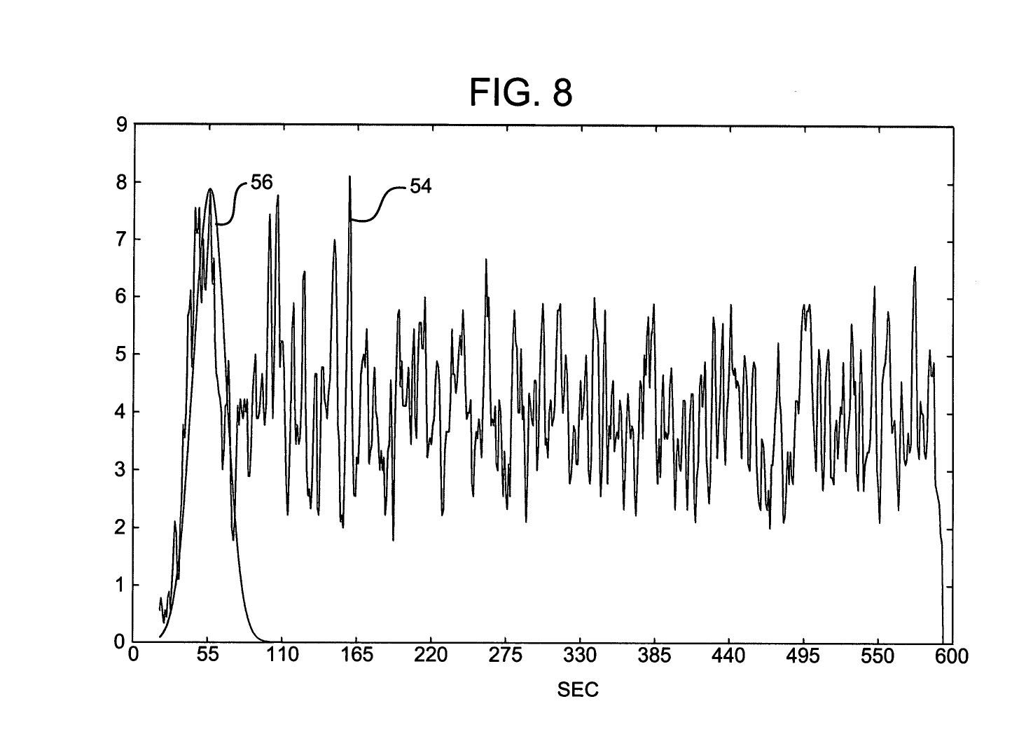

FIGS. 7 and 8 are schematic plots of the recurrence function and histogram, respectively, of a saturation signal in still another patient, in accordance with an embodiment of the present invention. The patient suffered from obstructive apnea, but not Cheyne-Stokes breathing. As a result, little or no band structure is seen in FIG. 7. A histogram 54 in FIG. 8 does not exhibit any meaningful periodicity, as shown by a parametric curve 56 that is fit to the histogram.

Other types of aberrant waveforms in the photoplethysmograph signal may correspond to motion artifacts. Motion is characterized by local maxima well above normal beat range (for example, at least twice the normal value). The prevalence of motion artifacts can be used in detecting movement, which indicate whether the patient is in a sleep or waking state. Alternatively or additionally, a motion sensor may be used to detect arousals.

Processor 32 may additionally extract other cardiorespiratory parameters from the signal, either directly or indirectly. For example, the processor may apply very-low-frequency filtering to the heart rate in order to detect heart rate modulations. Additionally or alternatively, the envelope of the signal may be processed in order to detect characteristics of vasomodulation, i.e., arterial dilation and constriction.

In some embodiments of the present invention processor 32 computes a respiration energy and/or rate characteristic based on high-frequency components of the signal. Respiratory sinus arrhythmia is a natural cycle of arrhythmia that occurs in healthy people through the influence of breathing on the flow of sympathetic and vagus impulses to the sinoatrial node in the heart. This effect may be used to calculate respiration from heart rate. Well-treated heart failure patients, however, are frequently under the control of cardiac pacemakers and often take beta-blockers and ACE inhibitors that suppress this phenomenon. High-frequency (10-30 cycles/min, i.e., 0.17-0.5 Hz) filtering of the photoplethysmograph signal enables the processor to determine respiration energy and/or rate characteristics in these cases, as well.

Very-low-frequency components of the characteristic can be indicative of a respiration modulation. Processor 32 can combine the various cardiac, respiratory and vasomodulation parameters described above in order to provide a general picture of cardiorespiratory effects, all on the basis of the photoplethysmograph signals.

Similar procedures to those described above can be applied to the detrended AC photoplethysmograph signal. One way to perform detrending is to replace the photoplethysmograph signal with its amplitude feature (maximum minus minimum signal). Other methods include subtracting a polynomial that approximates the signal, or using local maxima or local minima features. Following detrending, the processor applies very-low-frequency filtering followed by outlier rejection, and then computes the median vasomotion of each sequence.

The processor may perform similar analyses on heart rate and respiratory signals from other sources. Arousals can estimated from motion artifacts as described above or from other data if available (such as EEG alpha and beta frequencies, or scorer marking, or a motion sensor).

Information regarding sleep/wake state can be combined with the Cheyne-Stokes time to determine specific, cumulative Cheyne-Stokes time during sleep. The total Cheyne-Stokes time and percentage of Cheyne-Stokes time during sleep have prognostic value: a large percentage of Cheyne-Stokes time is associated with mortality and high levels of brain natriuretic peptide (BNP), which are associated with severity of heart failure. Furthermore, information about sleep time can be used to ensure that low Cheyne-Stokes duration is not associated with little or no sleep.

The information regarding the Cheyne-Stokes time can also be combined with the general picture of cardiorespiratory effects in order to provide some or all of the following combined information for each Cheyne-Stokes sequence during sleep: duration, median wavelength, median desaturation, median vasomotion, median heart rate modulation, median respiratory modulation, number of PVCs and other premature beats, and arousal index (e.g., number of arousals). Each of the above parameters can also be computed separately for REM sleep and NREM sleep.

### Clinical Application and Results

The methods described above for measuring and quantifying Cheyne-Stokes breathing and attendant heart failure prognosis provide the physician with a clear, immediate, visual indication of the patient's respiratory condition. They may be used conveniently in performing frequent checks on patient status, both at home and in the hospital. Additionally or alternatively, occasional checks of this sort may be used for risk stratification and screening. As explained above, these methods may be implemented using measurements made solely by pulse oximetry device 24, or alternatively in conjunction with other sensors, as in a multi-monitor polysomnography system, or in an implantable device, or using other types of sensors.

The above-mentioned US 2007/0213622 describes a study in which Cheyne-Stokes breathing characteristics were compared with heart failure status in tests of advanced heart failure patients. This study used the cumulative duration of Cheyne-Stokes breathing during a night's sleep as an indicator, and compared its sensitivity and specificity with measurements of Brain Natriuretic Peptide (BNP) in the patients' blood, which have been generally considered the best marker for heart failure status. The measurement of Cheyne-Stokes duration was found to compare favorably with BNP measurement as a prognostic indicator for heart failure.

The inventors in the present patent application performed a similar clinical study of the efficacy of Cheyne-Stokes cycle length as a prognostic indicator for heart failure. The cycle length (CL) was measured, using the techniques described above and illustrated in FIGS. 3-8, for a group of 109 heart failure patients. The median CL for this patient group was found to be 51 sec. High CL, above the median, was taken as a trial prognostic indicator for the patient group, for comparison with BNP level. The clinical progress of the patients' heart failure was monitored for a six-month period following the CL and BNP measurements in order to compare the prognostic value of these two indicators.

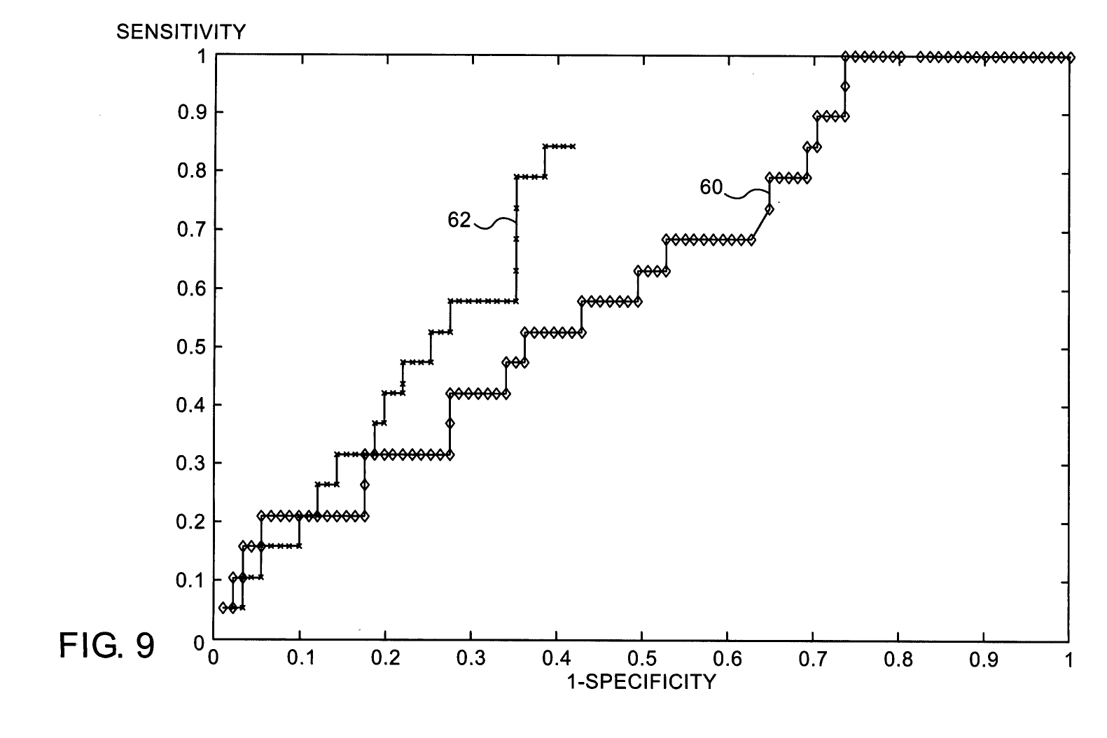

FIG. 9 is a receiver operating characteristic (ROC) plot, which schematically compares the sensitivity and specificity of BNP and of CL of Cheyne-Stokes breathing in predicting heart failure prognosis, in accordance with an embodiment of the present invention. An upper trace 62 is the ROC curve for Cheyne-Stokes CL≧51, while a lower trace 60 is the ROC curve for the BNP marker. To derive the results shown in the figure, both CL and BNP were tested against hospitalization and mortality of the patients over the six-month period of the study. The plot shows that the CL marker gave greater sensitivity and specificity in predicting which patients would be hospitalized or would die during the study period.

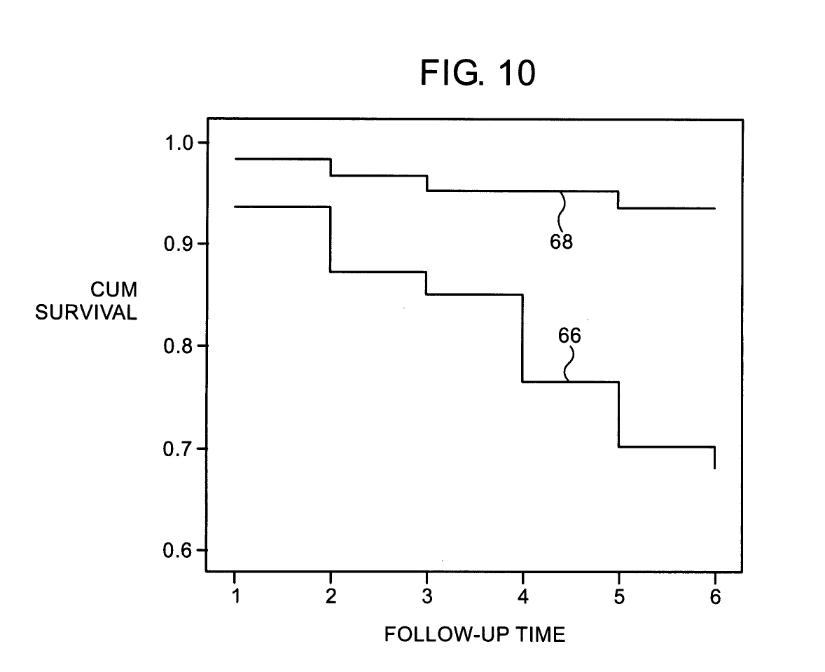

FIG. 10 is a Kaplan-Meier plot of a six-month survival function of the heart failure patients as a function of Cheyne Stokes CL, in accordance with an embodiment of the present invention. In this case, patients were classified into two groups: one group with CL≧51, and the other with CL<51. For the purpose of the plots in FIG. 10, the “survival” function corresponds to the number of patients who did not die and were not hospitalized over the study period. An upper trace 68 in the figure shows the survival rate for the group with CL<51, while a lower trace 66 shows the much lower survival rate for patients with CL≧51. The plot demonstrates the strength of CL as a prognostic marker.

The results of FIGS. 9 and 10 show that CL measurement based on photoplethysmographic monitoring during sleep may be used effectively for the prognosis of heart failure patients. As explained above, this sort of monitoring is simple to carry out in the patient's home or hospital bed and may be performed at regular intervals, at low cost and minimal discomfort to the patient. It provides physicians with an accurate prognostic indicator, which they can use in choosing the optimal treatment, such as determining whether a patient requires hospitalization, and adjusting treatment parameters (such as drug titration) based on changes in the patient's condition. For example, using the techniques described above, the physician may measure and quantify the patient's symptoms prior to initiating or making a change in treatment, and may repeat the measurement thereafter in order to assess the effectiveness of the treatment and possibly readjust treatment parameters.

As another alternative, the physician may fix a specific critical Cheyne-Stokes CL for individual patients, and then set a monitoring system to alarm whenever this CL is exceeded.

As noted earlier, although the embodiments described above relate mainly to signals captured by pulse oximetry device 24, the principles of the present invention may be applied to respiration signals captured by any other suitable type of sensor.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non limiting fashion.

### Prototype System

Following is a description of a prototype system which produces sleep analysis.

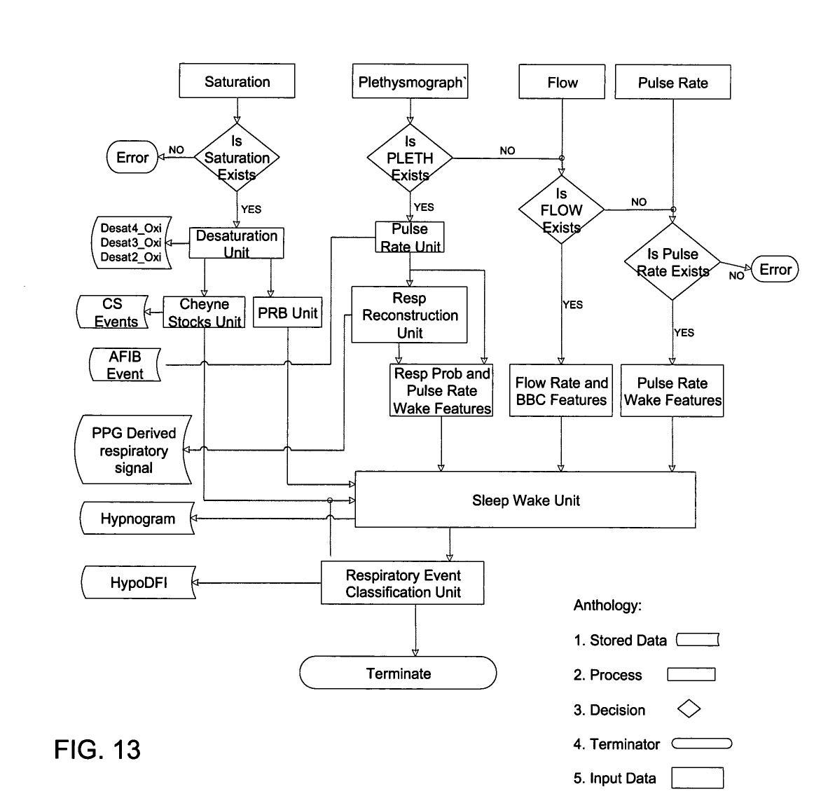

FIG. 13 depicts a high level block diagram of the prototype system. The block diagram shows sleep analysis from at least one of the following signals: saturation signal, pulse rate, PPG and respiration flow. The system comprises several analysis units.

A saturation unit which is responsible of desaturation event detection, Cheyne stokes respiration pattern classification and periodic respiration breathing (PRB).

A Plethysmograph unit which extracts location and magnitude of the PPG. Both are used in order to extract AFIB rhythms, respiratory rate and sleep wake features.

A flow unit for respiratory rate and breath by breath correlation (BBC) as features for sleep wake classification.

A pulse rate unit which calculates pulse rate slope calculation and pulse rate noise extraction for the determination of sleep wake epochs.

A sleep wake classification unit is utilized according to the input data; each feature is associated with probability for sleep and probability for wake and a Viterbi algorithm is performed for sleep wake hypnogram extraction.

A respiratory classification unit, receives hypnogram and desaturation events and classifies highly periodic desaturation events as central sleep apnea and also filter noisy wake desaturation events in order to produce respiratory events.

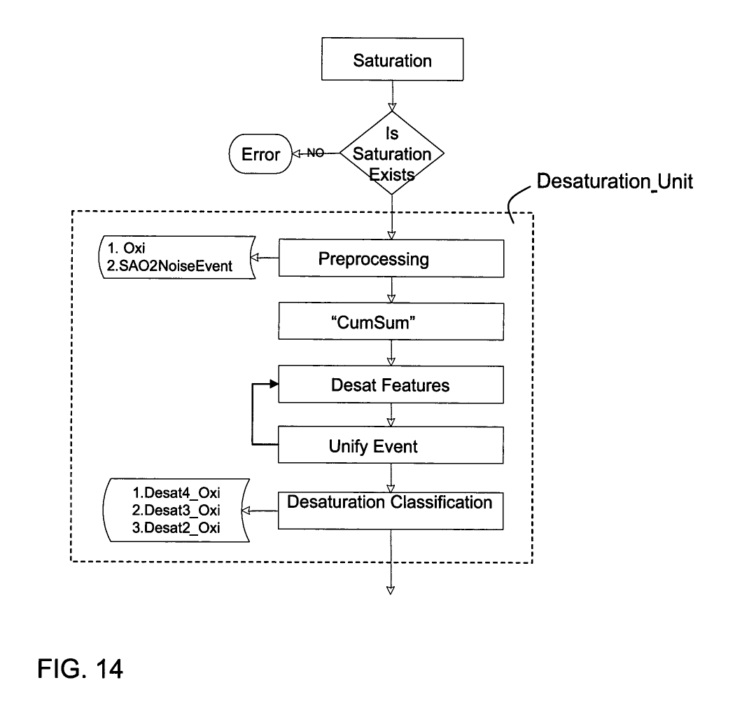

The saturation unit is further detailed in FIG. 14. The saturation unit can include several sub-units which operate as described above with reference to FIGS. 11A-B and 12A-C. The preprocessing subunit performs the preprocessing operation, as further detailed hereinabove, the CumSum subunit calculates and provides the derivative signal as further detailed hereinabove, the Desat Features subunit performs the saturation reduction thresholding procedure as further detailed hereinabove, the unification event subunit unifies intervals over the derivative signal as further detailed hereinabove, and the classification subunit outputs the desaturation features according to their classification.

In order to identify Cheyne Stokes breathing (CSB) of a patient, a collection of start and end points of 10 minutes of, e.g., desaturation 4% events, are fitted to a parametric model as further detailed hereinabove.

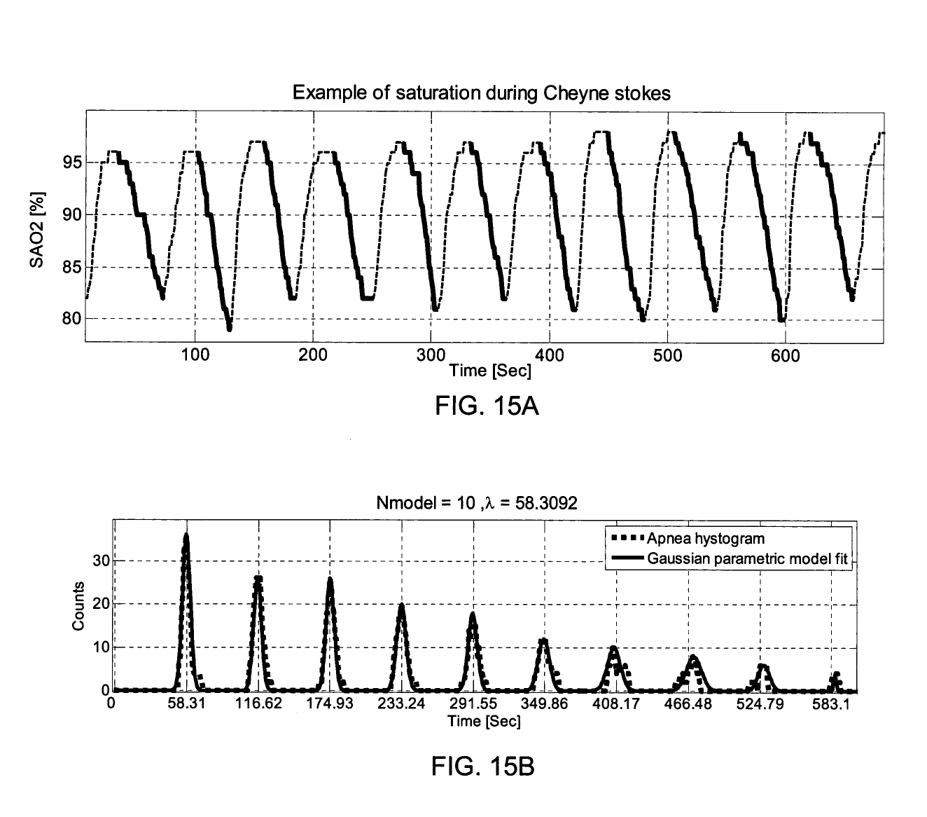

FIG. 15A shows example of saturation during Cheyne Stokes breathing, and FIG. 15B illustrates a parametric model fitting of 10 minutes of the Cheyne Stokes using a Gaussian kernel function. The cycle length is of about 58 seconds.

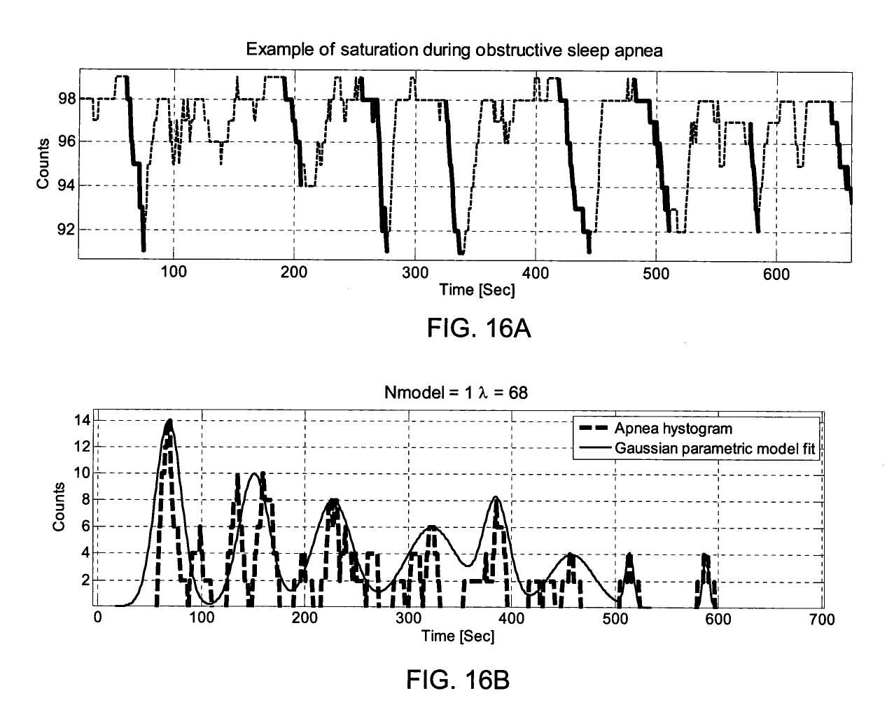

FIGS. 16A-B present the chaotic effect of obstructive sleep apnea. These features thus give a rapid quantitative assessment of the patient's respiratory conditions.

Once a collection of parameters for the parametric model are obtained, a set of rules are utilized to determine whether a set of apneas are associated with CSB. The rules can be based on the number of periodic desaturation, the width of the standard deviation and the perturbation needed in order to fit the parametric model to the data.

Two levels of CSB classification are initiated in case that suspected periodic of 3% or 4% detected CSB. In a first level, a set of four highly periodic desaturation events with cycle length in the zone of the average distance between consecutive apneas, are classified as CSB. In a second level a set of seven periodic desaturation events are classified as CSB.

The operation of saturation processing is finished once desaturation events, periodic respiratory breathing and Cheyne stokes breathing are extracted and ready to be used in sleep wake classification unit.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of diagnosis, comprising:
receiving a signal associated with respiration of a patient;
processing the signal so as to identify features that are indicative of beginning and end times of apnea episodes;
computing time shifts between recurrences of the features;
processing the time shifts so as to generate an output comprising a plurality of bands corresponding to different multiples of a cycle length of the apnea episodes.

2. The method according to claim 1, wherein the signal is indicative of variations in a blood oxygen saturation in a body of the patient due to the apnea episodes.

3. The method according to claim 2, wherein the signal is received from a photoplethysmographic sensor coupled to the body of the patient.

4. The method according to claim 1, wherein processing the signal comprises defining a threshold level, and identifying the features as points at which the signal crosses the threshold level.

5. The method according to of claim 1, further comprising extracting the cumulative sum of the first derivative of the signal to provide a derivative signal, wherein said processing comprises extracting slope and duration values from said derivative signal.

6. The method according to claim 1, wherein processing the time shifts comprises presenting a plot of the bands on a display.

7. The method according to claim 6, wherein presenting the plot comprises displaying the time shifts as a function of time over a monitoring period.

8. The method according to claim 1, wherein processing the time shifts comprises generating a histogram of the time shifts.

9. The method according to claim 8, wherein processing the time shifts comprises fitting a parametric function to the histogram so as to extract the cycle length from the histogram.

10. The method according to claim 9, wherein the parametric function comprises a sum of kernel functions.

11. The method according to claim 1, further comprising diagnosing a medical condition of the patient responsively to the cycle length.

12. The method according to claim 11, wherein the medical condition comprises a heart failure of the patient, and wherein diagnosing the medical condition comprises assessing a severity of the heart failure based on the cycle length.

13. The method according to claim 1, further comprising classifying said apnea episodes to central sleep apnea episodes and obstructive sleep apnea episodes.

14. Apparatus for diagnosis, comprising:
a sensor, which is configured to be coupled to a body of a patient and to output a signal associated with associated with respiration of the patient; and
a processor, which is coupled to process the signal so as to identify features that are indicative of beginning and end times of apnea episodes, to compute time shifts between recurrences of the features, and to process the time shifts so as to generate an output comprising a plurality of bands corresponding to different multiples of a cycle length of the apnea episodes.

15. The apparatus according to claim 14, wherein the signal is indicative of variations in a blood oxygen saturation in a body of the patient due to the apnea episodes.

16. The apparatus according to claim 15, wherein the sensor comprises a photoplethysmographic sensor.

17. The apparatus according to claim 15, wherein the processor is configured to define a threshold level, and to identify the features as points at which the blood oxygen saturation crosses the threshold level.

18. The apparatus according to claim 14, and comprising a display, wherein the processor is coupled to present a plot of the bands on the display.

19. The apparatus according to claim 18, wherein the plot shows the time shifts as a function of time over a monitoring period.

20. The apparatus according to claim 14, wherein the output comprises a histogram of the time shifts.

21. The apparatus according to claim 20, wherein the processor is configured to fit a parametric function to the histogram so as to extract the cycle length from the histogram.

22. The apparatus according to claim 21, wherein the parametric function comprises a sum of kernel functions.

23. The apparatus according to claim 14, wherein the processor is configured to provide an indication of a severity of a heart failure of the patient based on the cycle length.

24. A computer software product, comprising a computer-readable medium in which program instructions are stored, which instructions, when read by a computer, cause the computer to receive a signal associated with respiration of a patient, to process the signal so as to identify features that are indicative of beginning and end times of apnea episodes, to compute time shifts between recurrences of the features, and to process the time shifts so as to generate an output comprising a plurality of bands corresponding to different multiples of a cycle length of the apnea episodes.
